(19) United States
(12) Patent Application Publication
(10) Pub. No.: US 2004/0229940 A1
(43) Pub. Date: Nov. 18, 2004

(54) Analeptic and antidepressant combinations

(75) Inventors: Howard A. Hassman, Moorestown, NJ (US); Robert T. Hrubiec, Unionville, PA (US); Rodney J. Hughes, Kennett Square, PA (US); Jeffry L. Vaught, Glenmoore, PA (US)

Correspondence Address: CEPHALON, INC., 145 BRANDYWINE PARKWAY, WEST CHESTER, PA, 19380-4245, US

(73) Assignee: Cephalon Inc, West Chester, PA

(21) Appl. No.: 10/843,988

(22) Filed: May 12, 2004

Related U.S. Application Data
(60) Provisional application No. 60/469,945, filed on May 13, 2003.

(51) Int. Cl. A61K 31/343; A61K 31/165; A61K 31/137

(52) U.S. Cl. 514/469; 514/649; 514/617

(57) ABSTRACT

Compositions and methods for the treatment of depressive disorders through the administration of modafinil with antidepressants.

### BACKGROUND OF THE INVENTION

1. Modafinil

Modafinil, C₁₅H₁₅NO₂S, also known as 2-(benzhydrylsulfinyl) acetamide, or 2-[(diphenylmethyl) sulfinyl] acetamide, is a synthetic acetamide derivative with wake-promoting activity, the structure of which has been described in French Patent No. 78 05 510 and in U.S. Pat. No. 4,177,290 ('290), and which has been approved by the United States Food and Drug Administration for use in the treatment of excessive daytime sleepiness associated with narcolepsy. A method of preparation of a racemic mixture is described in the '290 patent and a method of preparation of a levorotatory isomer is described in U.S. Pat. No. 4,927,855 (both incorporated herein by reference). The levorotatory isomer is reported to be useful for treatment of hypersomnia, depression, Alzheimer's disease and to have activity towards the symptoms of dementia and loss of memory, especially in the elderly.

The primary pharmacological activity of modafinil is to promote wakefulness. Modafinil promotes wakefulness in rats (Touret et al., 1995; Edgar and Seidel, 1997), cats (Lin et al., 1992), canines (Shelton et al., 1995) and non-human primates (Hernant et al, 1991) as well as in models mimicking clinical situations, such as sleep apnea (English bulldog sleep disordered breathing model) (Panckeri et al, 1996) and narcolepsy (narcoleptic canine) (Shelton et al, 1995).

Modafinil has also been described as an agent with activity in the central nervous system, and as a useful agent in the treatment of Parkinson's disease (U.S. Pat. No. 5,180,745); in the protection of cerebral tissue from ischemia (U.S. Pat. No. 5,391,576); in the treatment of urinary and fecal incontinence (U.S. Pat. No. 5,401,776); and in the treatment of sleep apneas and disorders of central origin (U.S. Pat. No. 5,612,379). U.S. Pat. No. 5,618,845 describes modafinil preparations of a defined particle size less than about 200 microns. In addition, modafinil may be used in the treatment of eating disorders, or to promote weight gain or stimulate appetite in humans or animals (U.S. Pat. No. 6,455,588, incorporated herein by reference), or in the treatment of attention deficit hyperactivity disorder (ADHD) (U.S. Pat. No. 6,346,548, incorporated herein by reference), or fatigue, especially fatigue associated with multiple sclerosis (U.S. Pat. No. 6,488,164, incorporated herein by reference).

Modafinil has been shown to be effective in treating narcolepsy, sleepiness, excessive sleepiness (e.g., sleepiness associated with disorders of sleep and wakefulness), excessive daytime sleepiness associated with narcolepsy, Parkinson's disease, urinary incontinence, multiple sclerosis fatigue, ADHD, Alzheimer's disorder, sleep apnea, obstructive sleep apnea, depression, and ischemia.

Narcolepsy is a chronic disorder characterized by intermittent sleep attacks, persistent, excessive daytime sleepiness and abnormal rapid eye movement (“REM”) sleep manifestations, such as sleep-onset REM periods, cataplexy, sleep paralysis and hypnagogic hallucinations, or both. Most patients with narcolepsy also have disrupted nocturnal sleep. Pathological somnolence, whether due to narcolepsy or other causes, is disabling and potentially dangerous. Causes of pathological somnolence, other than narcolepsy, include chronic sleep loss; sleep apnea; and other sleep disorders. Whether due to narcolepsy or other causes, pathological somnolence produces episodes of unintended sleep, reduced attention, and performance errors. Consequently, it is linked to a variety of transportation and industrial accidents. A therapeutic agent that reduces or eliminates pathological somnolence would have important implications not only for individual patients, but also for public health and safety.

Other uses of modafinil have been presented. U.S. Pat. No. 5,180,745 discloses the use of modafinil for providing a neuroprotective effect in humans, and in particular for the therapy of Parkinson's disease. The levorotatory form of modafinil, i.e., (−) benzhydrylsulfinyl-acetamide, may have potential benefit for therapy of depression, hypersomnia and Alzheimer's disease (U.S. Pat. No. 4,927,855). European Published Application 547952 discloses the use of modafinil as an anti-ischemic agent. European Published Application 594507 discloses the use of modafinil to treat urinary incontinence.

U.S. Pat. No. RE37,516 discloses pharmaceutical compositions having a defined particle size, and in particular compositions wherein 95% of the cumulative total of the effective amount of modafinil particles in the composition have a diameter less than about 200 microns.

2. Antidepressants

Antidepressants, including selective serotonin reuptake inhibitors (SSRIs) have become first choice therapeutics in the therapy of depression, certain forms of anxiety and social phobias. In some instances, SSRIs can be more favored because they are effective, well tolerated and have a favorable safety profile compared to the classic tricyclic antidepressants.

However, there can be problems associated with any anti-depressant. Current antidepressant therapy can exhibit a delayed onset and modest proportion in achieving response or remission. For example, the response at 6 weeks to the selective serotonin reuptake inhibitor (SSRI) fluoxetine is about 50%. Remission rates with SSRIs at 8 weeks are about 35%. Delayed, incomplete and lack of response of a major depressive disorder to antidepressant therapy can be problematic for numerous reasons, including premature treatment discontinuation. Sometimes symptoms even worsen during the first weeks of therapy. In other cases, non-compliance can be related to side effects, including sexual dysfunction.

Fatigue and excessive sleepiness are among the symptoms of a major depressive disorder, and can be adverse experiences associated with antidepressant therapy and are often residual symptoms inadequately treated with SSRI antidepressant therapy.

In addition, patients sometimes suffer side effects associated with antidepressant therapy and withdrawal of antidepressant therapy.

Because residual symptoms to antidepressant therapy predisposes patients with depression to a greater risk of relapse and greater probability of recurrence, rapid achievement of remission is an important consideration in choosing the most appropriate treatment strategy.

New therapies that address one or more of these problems are needed.

### SUMMARY OF THE INVENTION

In one embodiment, a composition can include therapeutically effective amounts of two or more active compounds, including but not limited to 1) an analeptic, and 2) an antidepressant. The actives can be combined together and optionally include a pharmaceutically acceptable carrier or the actives can be administered separately.

In one embodiment, the present invention includes a method of treating a depressive disorder by providing a pharmacologically active composition or a pharmacologically active combination. The pharmacologically active composition or combination can include a therapeutically effective amount of an analeptic and one or more antidepressants.

### DETAILED DESCRIPTION OF THE INVENTION

1. Analeptic Agents

Analeptics are drugs that principally act as or are used as a central nervous system stimulant. Preferred for use in the practice of the invention are analeptics that operate on the sleep-wake centers of the brain and that lack the pharmacological effects of amphetamines. Preferred analeptic agents have the pharmacological profile of modafinil. Thus, in a preferred embodiment of the invention, the analeptic used in the practice of the invention is Provigil® (modafinil).

2. Antidepressants

Useful antidepressants include but are not limited to tricyclic antidepressants (“TCAs”), Selective Serotonin Reuptake Inhibitors (“SSRIs”), Serotonin and Noradrenaline Reuptake Inhibitors (“SNRIs”), Dopamine Reuptake Inhibitors (“DRIs”), Noradrenaline Reuptake Inhibitors (“NRUs”), Dopamine, Serotonin and Noradrenaline Reuptake Inhibitors (“DSNRIs”) and Monoamine Oxidase Inhibitors (“MAOIs) including reversible inhibitors of monoamine oxidase type A (RIMAs).

In certain embodiments, a suitable antidepressant can include, but is not limited to, one or more of the following antidepressants: adatanserin hydrochloride; adinazolam; adinazolam mesylate; alaproclate; aletamine hydrochloride; amedalin hydrochloride; amitriptyline hydrochloride; amoxapine; aptazapine maleate; azaloxan fumarate; azepindole; azipramine hydrochloride; bipenarnol hydrochloride; bupropion hydrochloride; butacetin; butriptyline hydrochloride; caroxazone; cartazolate; ciclazindol; cidoxepin hydrochloride; cilobamine mesylate; citalipram; clodazon hydrochloride; clomipramine hydrochloride; cotinine fumarate; cyclindole; cypenamine hydrochloride; cyprolidol hydrochloride; cyproximide; daledalin tosylate; dapoxetine hydrochloride; dazadrol maleate; dazepinil hydrochloride; desipramine hydrochloride; dexamisole; deximafen; dibenzepin hydrochloride; dioxadrol hydrochloride; dothiepin hydrochloride; doxepin hydrochloride; duloxetine hydrochloride; eclanamine maleate; encyprate; etoperidone hydrochloride; fantridone hydrochloride; fehmetozole hydrochloride; fenmetramide; fezolamine fumarate; fluotracen hydrochloride; fluoxetine; fluoxetine hydrochloride; fluparoxan hydrochloride; gamfexine; guanoxyfen sulfate; imafen hydrochloride; imiloxan hydrochloride; imipramine hydrochloride; indeloxazine hydrochloride; intriptyline hydrochloride; iprindole; isocarboxazid; ketipramine fumarate; lofepramine hydrochloride; lortalamine; maprotiline; maprotiline hydrochloride; melitracen hydrochloride; milacemide hydrochloride; minaprine hydrochloride; mirtazapine; moclobemide; modaline sulfate; napactadine hydrochloride; napamezole hydrochloride; nefazodone hydrochloride; nisoxetine; nitrafudam hydrochloride; nomifensine maleate; nortriptyline hydrochloride; octriptyline phosphate; opipramol hydrochloride; oxaprotiline hydrochloride; oxypertine; paroxetine; phenelzine sulfate; pirandamine hydrochloride; pizotyline; pridefine hydrochloride; prolintane hydrochloride; protriptyline hydrochloride; quipazine maleate; rolicyprine; seproxetine hydrochloride; sertraline hydrochloride; sibutramine hydrochloride; sulpiride; suritozole; tametraline hydrochloride; tampramine fumarate; tandamine hydrochloride; thiazesim hydrochloride; thozalinone; tomoxetine hydrochloride; trazodone hydrochloride; trebenzomine hydrochloride; trimipramine; trimipramine maleate; venlafaxine hydrochloride; viloxazine hydrochloride; zimeldine hydrochloride; zometapine.

In certain embodiments, the antidepressant includes citalipram, fluoxetine, fluoxetine hydrochloride, paroxetine, paroxetine hydrochloride, and/or clomipramine hydrochloride, with citalipram, paroxetine, fluoxetine and fluoxetine hydrochloride preferred, with citalipram most preferred.

Other drugs which are useful in treating depressive disorders, e.g., tiagabine, can also be used in the practice of the invention.

3. Variants, Analogs, Salts, Different Forms

Antidepressants not listed above, including but not limited to structural analogs of the above compounds, that are safe and effective, are also useful in the practice of the invention.

Included within the scope of this invention are the various individual stereoisomers, including diastereomers and enantiomers (e.g., the L and/or R-isomer of modafinil) as well as mixtures thereof. In addition, compounds useful in this invention also include any pharmaceutically acceptable salts, for example: alkali metal salts, such as sodium and potassium; ammonium salts; monoalkylammonium salts; dialkylammonium salts; trialkylammonium salts; tetraalkylammonium salts; and tromethamine salts. Hydrates, solvates, and polymorphs of the compounds described above are included within the scope of this invention. Combinations of analeptics and of antidepressants can also be employed. The compounds can be substantially pure or mixed with other ingredients.

4. Depressive Disorders

The invention is useful in the treatment of depression, including mild to severe or acute depression, that may be caused by any of a number of factors, including, for example, depression associated with alcohol or drug abuse. The invention is also useful in the treatment of other disorders for which antidepressants are sometimes prescribed. These include, for example, anxiety, stress, social phobia, panic, obsession, compulsive behavior, pain (e.g., neuropathic and inflammatory pain) etc. Such disorders, for which antidepressants have been shown to have clinically beneficial effects, are herein referred to collectively as “depressive disorders.”

5. Therapeutically Effective Amounts of Analeptics and Antidepressants

In one embodiment of the present invention, an amount of analeptic, e.g. modafinil, administered to a patient can include 5, 10, 15, 20, 30, 40, 50, 60, 70, 75, 80, 90, 100, 200, 300 and/or 400 mg. of modafinil, or combinations thereof. Typically, modafinil can be administered in 50, 75, 100 and 200 mg. amounts. However, when used in combination with one or more antidepressants, as described herein, the amount of modafinil necessary to alleviate all or a portion of the symptoms associated with antidepressant therapy can be reduced. Accordingly, one embodiment of the present invention includes 100 mg. or less of modafinil when administered with an antidepressant, either as a combined unit dose with the antidepressant or as a separate dose. A single unit dose containing both modafinil and an antidepressant is a preferred composition of the present invention, as described below.

Typically, one or more antidepressants can be administered in the amounts known to be effective for each antidepressant. More specifically, in the present invention, an antidepressant can be administered in an amount effective to alter the depressive state of an animal subject, i.e., the amount of antidepressant that would be administered to the animal subject if the antidepressant was administered alone. Suitable amounts can include 5, 10, 15, 20, 30, 40, 50, 60, 70, 80, 90, 100, 200, 300 and/or 400 mg. of a particular antidepressant or combinations thereof. However, in the present invention, when used in combination with one or more analeptics such as modafinil, the overall amount of an administered antidepressant can be reduced by 10%, 20%, 30%, 40%, 50%, 60%, 70% or 80%, while still providing an antidepressant effect. Accordingly, one embodiment of the present invention includes administering less than an amount of antidepressant relative to the amount of antidepressant administered to an animal subject if administered alone.

Generally, for daily oral doses of active compounds, the combined total of one or more analeptics and one or more antidepressants will be from about 0.01 mg/kg per day to about 2000 mg/kg per day. It is expected that IV doses in the range of about 1 to 1000 mg/cm³ per day will be effective.

In some embodiments of the present invention, the respective weight ratio of analeptic to antidepressant can be from 0.01:1 to 1:1 to 100:1, possibly 1000:1 In some embodiments the weight ratio can be 1:1 to 7:1 or 10:1, most preferably 1:1 to 5:1.

A dosage form containing an above described amount of an analeptic (e.g., modafinil) and one or more antidepressants can provide to a patient improved fatigue symptoms, as well as improve waking functioning, as demonstrated by the effects of fatigue, energy, alertness and cognitive function (e.g. psychomotor retardation).

6. Preparation of a Composition of the Present Invention

To prepare a pharmaceutical composition of this invention, an analeptic, including but not limited to modafinil, and an antidepressant, including but not limited to one or more of the antidepressants described above, can be intimately admixed. The mixture can further optionally include a pharmaceutical carrier according to conventional pharmaceutical compounding techniques, which carrier may take a wide variety of forms depending on the form of preparation desired for administration, e.g., oral, by suppository, or parenteral. The amount of each active component in the composition can correspond to the amounts described above. Pharmaceutically acceptable carriers include, e.g., stabilizers binders, fillers, disintegrants, lubricants, coatings, sweeteners, flavors, colors, diluents, etc. Such a composition, when used for the therapy of a depressive disorder preferably can include therapeutically effective amounts of an analeptic and antidepressant.

In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques.

For parenterals, the carrier will usually comprise sterile water, though other ingredients, for example, for purposes such as aiding solubility or for preservation, may be included. Injectable suspensions may also be prepared in which case appropriate liquid carriers, suspending agents and the like may be employed.

In one embodiment, a pharmaceutical composition of the present invention can be administered in a tablet or capsule form or other suitable unit dose form. A tablet or capsule of the present invention can contain one or more of the following inactive ingredients: lactose hydrous, pregelatinized starch, microcrystalline cellulose, sodium starch glycolate, magnesium stearate, purified water, carnauba wax, hydroxypropyl methylcellulose, titanium dioxide, polyethylene glycol, synthetic iron oxide, and polysorbate 80, etc.

Accordingly, a pharmaceutical compositions herein will contain, per dosage unit, e.g., tablet, capsule, powder injection, teaspoonful, suppository and the like from about 5 to about 1000 mg, or more, of an analeptic and antidepressant. In one embodiment of the invention, each single dosage unit (or unit dose) includes both an amount of an analeptic and an amount of an antidepressant. In such embodiment, it is not necessary that each single dosage unit include an effective amount so long as the total amount of drug administered to a patient is an effective amount of each. Therefore, for example, a patient may require 2 or more single dosage units to receive effective amounts of both agents.

When administered, the formulations of the invention are applied in pharmaceutically acceptable amounts and in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

Dosage may be adjusted appropriately to achieve desired drug levels, locally or systemically. As noted above, generally, daily oral doses of active compounds will be from about 0.01 mg/kg per day to 2000 mg/kg per day. In the event that the response in a subject is insufficient at such doses, even higher doses (or effective higher doses by a different, more localized delivery route) may be employed to the extent that patient tolerance permits. Continuous IV dosing over, for example 24 hours or multiple doses per day is contemplated to achieve appropriate systemic levels of compounds.

A variety of administration routes are available. The particular mode selected will depend of course, upon the particular drug selected, the severity of the disease state(s) being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, or infusion.

The compositions may conveniently be presented in unit dosage form and may be prepared by any of the methods well known in the art of pharmacy. In general, the compositions are prepared by uniformly and intimately bringing the compounds into association with a liquid carrier, a finely divided solid carrier, or both, and then, if necessary, shaping the product.

Compositions suitable for oral administration may be presented as discrete units such as capsules, cachets, tablets, or lozenges, each containing a predetermined amount of the active compound. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the active compounds of the invention, increasing convenience to the subject and the physician. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

Another embodiment of the present invention provides a kit or device which can facilitate the administration of an amount of an analeptic and an antidepressant to treat a depressive disorder. Specifically, a kit according to the present invention includes at least one dosage form containing an analeptic, including but not limited to modafinil, and a separate dosage form containing at least one antidepressant. One suitable kit of the present invention includes a blister pack having a unit dose of modafinil and a separate unit dose of an antidepressant. Most preferably, the unit dose of modafinil includes a 50, 75, 100 or 200 mg. tablet of modafinil and the unit dose of antidepressant includes a 10, 20, 30, 40 or 50 mg. tablet of antidepressant. The kit or device can also include instructions concerning administration of the analeptic and antidepressant. Preferably, the instructions provide administration guidance according to one or more of the administration schemes set forth below.

The analeptic and/or antidepressant can be in any suitable dosage form, including but not limited to solid dosage forms including tablets, capsules, pills, troches, cachets, and the like, and/or liquid dosage forms such as an oral elixir or an IV fluid. The dosage form of the analeptic can be the same type or a different type than the antidepressant.

In yet another embodiment, the present invention includes a transdermal drug delivery system (“TDDS”). A TDDS suitable for use with the invention in patch form typically contains at least: (1) a backing layer and (2) a carrier formulated with an effective amount of an antidepressant and optionally modafinil.

Preferred patches include (1) the matrix type patch; (2) the reservoir type patch; (3) the multi-laminate drug-in-adhesive type patch; and (4) the monolithic drug-in-adhesive type patch. These patches are generally available commercially.

For practice of the invention, the matrix type and the drug-in-adhesive type patches are especially preferred. The more preferred drug-in-adhesive patch is the monolithic type.

Transdermal drug delivery systems other than standard patches can also be used. These include, for example, osmotic pump systems, ultrasonic systems, ointments, pastes, gels, medicated powders, creams, lotions, aerosols, sprays, foams, medicated adhesives and the like.

7. Method of Treatment/Therapy

A. Administration Schemes and Timing of Treatment of an Analeptic and Antidepressant

An analeptic and an antidepressant can be combined together into a single unit dose, but can also be administered separately as two or more distinct doses.

Thus, in some embodiments of the invention, a treatment of a disorder related to depression can be through the use of separate dosage forms—one or more analeptic doses and one or more antidepressant doses. Accordingly, a dose of an analeptic can be administered at a different time relative to the antidepressant dose or simultaneously (i.e., analeptic dose administration within less than 1 hour before or after administration of the antidepressant). However, if simultaneous administration is desired, the administration of the analeptic and antidepressant can also be through the use of a single unit dose including both an analeptic and antidepressant.

In patients that are beginning antidepressant therapy, i.e. patients that are substantially free of antidepressants or patients that have been free of antidepressant therapy for about 1 week, 2 weeks, more preferably about 4 or more weeks, the dosage form containing the analeptic can be administered before and/or at about the same time as an initial administration of the antidepressant. In such an embodiment, one or more administrations of an analeptic can be within 72 hours, preferably within 48 hours, more preferably within 24 hours, most preferably within 1 hour or moments before an initial administration/dosing of an antidepressant. After the initial administration of the analeptic and antidepressant, subsequent dosings of the analeptic and antidepressant can continue at a typical rate, e.g., 50, 75, 100 to 200 mg. doses of modafinil per day and 10, 20, 30, 40, 50 mg. of antidepressant per day. Further, after the initial administration of the antidepressant, the dosings of the analeptic and antidepressant can be in separate dosage forms or in a single unit dose. However, if a dose of an analeptic is to be administered before a subsequent dose of an antidepressant, separate dosage forms for each are preferred.

Additionally, in patients that are substantially free of antidepressants, the initial administration of the analeptic can coincide with or be nearly simultaneous with the initial administration of an antidepressant. This can be accomplished through the use of separate dosage forms of an analeptic and antidepressant which can then be administered together simultaneously (i.e., within 1 hour or less, before or after the antidepressant) or through the use of a single unit dose including both an analeptic and an antidepressant, as noted above.

Further, an analeptic, including but not limited to modafinil, can also be administered to a patient that has already received at least an initial dose of an antidepressant. In one embodiment, the initial administration of an analeptic can be within 72 hours, preferably within 48 hours, more preferably within 24 hours, most preferably within 1 hour or within moments after the initial administration of an antidepressant. In this timing scheme, modafinil is administered at about the same time as an antidepressant, but subsequent to at least one administration of an antidepressant. After the initial dosing of an analeptic, the dosing of the analeptic and antidepressant can continue in a typical manner. In one particularly preferred embodiment, initial administration of an analeptic and subsequent administrations of an analeptic can be accomplished through the use of a single unit dose including both an analeptic and an antidepressant.

In a further embodiment, initial administration of an analeptic to a patient can occur and/or continue after antidepressant therapy has ended. Preferably, this is accomplished by administering an amount of the analeptic to the patient and the administration of which can continue for 1, 2, 5, 10, 20, or 30 days, or more, after antidepressant therapy cessation.

In embodiments where the analeptic and antidepressant are in separate dosage forms, the administration of the analeptic can preferably occur within moments, or in less than 1 hour, or less than 5 hours, or less than 24 hours or less than 48 hours, or less than 72 hours before or after administration of the antidepressant, unless otherwise indicated by a particular method of treatment below.

B. Depressive Disorder Treatment Generally

In one embodiment, the present invention includes a method of treating a depressive disorder by providing a pharmacologically active composition to a subject in need of the composition, preferably a human subject. The pharmacologically active composition can include an amount of an analeptic, preferably a therapeutically effective amount of an analeptic, and one or more antidepressants. The composition can optionally further include a pharmaceutically acceptable carrier, as described above. The pharmacologically active composition can then be administered to an animal subject.

The analeptics and other drugs can be administered to a patient simultaneously or at different times, as described above, and can follow one or more of the administration schemes set forth above.

The pharmacologically active composition and/or combination can be administered via any acceptable route, including but not limited to orally, nasally, rectally, intravenously, epidurally, intraperitoneally, subcutaneously, intramuscularly or intrathecally.

In one embodiment, the administered analeptic is modafinil or a salt thereof. In another embodiment, the antidepressant can include one or more of the antidepressants described above, including but not limited to citalipram, fluoxetine and/or paroxetine.

In one embodiment, one or more analeptics are in a single unit dose form and one or more antidepressants are in a separate unit dose form. Each unit dose form can be administered simultaneously or at different times, according to one or more of the administration schemes described above. In a preferred embodiment, the pharmacologically active composition contains both an analeptic and an antidepressant in a single unit dose form.

In a further embodiment, a therapy method for treating a depressive disorder in an animal subject includes topically applying a pharmaceutically acceptable drug formulation having at least one antidepressant to the skin of an animal, wherein the pharmaceutically acceptable drug formulation is contained in a patch. An additional effective amount of one or more analeptics, including but not limited to modafinil, can be provided to the animal, either in a matrix included in the patch or via administration of the analeptic through any other acceptable route, including but not limited to orally, nasally, rectally, intravenously, epidurally, intraperitoneally, subcutaneously, intramuscularly or intrathecally.

Definitions

“Particle,” as used herein, refers to an aggregated physical unit of the acetamide compound, i.e., a piece or a grain of acetamide.

As used herein, “about” means plus or minus ten percent of the indicated value, such that “about 20 mg” indicates 18 to 22 mg.

As used herein, “consisting essentially of” refers to excluding other active ingredients but including excipients and additional amounts of the active ingredient to account for degradation or otherwise.

An “effective amount,” as used herein, is an amount of modafinil and/or antidepressant that is effective for treating a depressive state, i.e., an amount of modafinil and/or antidepressant that is able to reduce, alleviate or eliminate certain symptoms associated with depression and/or antidepression therapy.

A “pharmaceutical composition,” as used herein, means a medicament for use in treating a mammal that comprises modafinil prepared in a manner that is appropriate for administration to a mammal. A pharmaceutical composition according to the invention may also, but does not of necessity, include a non-toxic pharmaceutically acceptable carrier. A pharmaceutical composition can also include bulk active modafinil for use in preparing dosage forms. A pharmaceutical composition can also include modafinil in combination with another active, preferably and antidepressant, more preferably an SSRI.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. Further, the contents of all references cited herein are hereby incorporated by reference.

What is claimed is:

1. A pharmaceutical composition comprising an analeptic and an antidepressant.

2. The composition of claim 1 wherein the analeptic comprises modafinil and the antidepressant is selected from the group consisting of tricyclics, selective serotonin reuptake inhibitors, serotonin and noradrenaline reuptake inhibitors, monoamine oxidase inhibitors, and monoamine oxidase type A.

3. The composition of claim 2 wherein the antidepressant is selected from the group consisting of citalipram, fluoxetine, fluoxetine hydrochloride, paroxetine, paroxetine hydrochloride, and clomipramine hydrochloride.

4. The composition of claim 3 wherein the composition is a single unit dose.

5. The composition of claims 2 or 3 wherein the amount of antidepressant includes 5, 10, 15, 20, 30, 40, 50, 60, 70, 80, 90, 100, 200, 300 or 400 mg of antidepressant.

6. The composition of claim 5 wherein the amount of modafinil includes 5, 10, 15, 20, 30, 40, 50, 60, 70, 75, 80, 90, 100, 200, 300 or 400 mg of modafinil.

7. A method for treating a depressive disorder in a patient comprising administering a pharmacologically active analeptic and a pharmacologically active antidepressant to a patient to treat a depressive disorder wherein the administration of the analeptic is prior to or at about the same time as the administration of the antidepressant.

8. The method according to claim 7 wherein the analeptic comprises modafinil.

9. The method of claim 8 wherein the antidepressant is selected from the group consisting of tricyclics, selective serotonin reuptake inhibitors, serotonin and noradrenaline reuptake inhibitors, monoamine oxidase inhibitors, and monoamine oxidase type A.

10. The method of claim 9 wherein the antidepressant is selected from the group consisting of citalipram, fluoxetine, fluoxetine hydrochloride, paroxetine, paroxetine hydrochloride, and chomipramine hydrochloride.

11. The method according to claim 10 wherein the antidepressant is citalipram, fluoxetine or fluoxetine hydrochloride.

12. The method of claim 8 wherein the modafinil and antidepressant are combined in a single unit dose form.

13. The method of claim 8 wherein the amount of antidepressant includes 5, 10, 15, 20, 30, 40, 50, 60, 70, 80, 90, 100, 200, 300 or 400 mg of antidepressant.

14. The method of claim 8 wherein the amount of modafinil includes 5, 10, 15, 20, 30, 40, 50, 60, 70, 75, 80, 90, 100, 200, 300 or 400 mg of analeptic.
